(19) United States
(12) Patent Application Publication
(10) Pub. No.: US 2021/0077360 A1
(43) Pub. Date: Mar. 18, 2021

(54) SOLID POWDER COSMETIC AND METHOD FOR PRODUCING SOLID POWDER COSMETIC

(71) Applicant: TOKIWA CORPORATION, Gifu (JP)

(72) Inventor: Erika KAKEHI, Saitama (JP)

(73) Assignee: TOKIWA CORPORATION, Gifu (JP)

(21) Appl. No.: 16/959,550

(22) PCT Filed: Dec. 17, 2018
(86) PCT No.: PCT/JP2018/046366

§ 371 Date: Jul. 1, 2020

(30) Foreign Application Priority Data
Jan. 9, 2018 (JP) 2018-001399

(51) Int. Cl. A61K 8/02; A61K 8/891; A61K 8/92; A61Q 1/12; A61Q 1/10; A61Q 1/08

(52) U.S. Cl. CPC A61K 8/022; A61K 8/891; A61K 8/92; A61K 2800/60; A61Q 1/10; A61Q 1/08; A61Q 1/12

(57) ABSTRACT

A solid powder cosmetic is a solid powder cosmetic containing a powder component and an oily component, the contents of the powder component and the oily component are 70% to 95% by mass and 5% to 30% by mass, respectively, based on the total amount of the solid powder cosmetic, and the oily component contains (A) a hardened castor oil fatty acid ester and (B) a heavy liquid isoparaffin.

### TECHNICAL FIELD

The present invention relates to a solid powder cosmetic and a method for producing a solid powder cosmetic.

### BACKGROUND ART

A solid powder cosmetic is composed of a powder component and an oily component, and solid powder cosmetics are utilized in makeup cosmetics such as eye shadows, blushers, and foundations. In a case in which an applied color is required to exhibit a pearly feeling, a lustrous powder or the like are used as the powder component.

From the viewpoint of the convenience in transportation upon carrying, solid powder cosmetics are often used in the form of being accommodated in compact vessels. Regarding a method for producing a solid powder cosmetic in such a form, a dry production method of mixing a powder component and an oily component, filling a mold with the mixture, and forming the mixture by pressing; and a wet production method of mixing a powder component and an oily component in a solvent to obtain a slurry form, filling a mold with the slurry thus obtained, lightly pressing the slurry, subsequently drying and removing the solvent, and thereby forming the residue, are known.

A solid powder cosmetic obtainable by a wet production method tends to exhibit good spreading at the time of application and to provide excellent feeling of use such as adherence. However, there have been occasions in which the brightness of the surface of a formed cosmetic increases, consequently the cosmetic shades into white, the applied color is not easily conveyed to the buyer from the color in appearance, and at the time of use, the color difference between the surface color of an unused portion and the surface color of a used portion becomes large.

In Patent Literature 1 described below, there has been suggested a technique of incorporating a silica having an oil absorption amount of 100 ml/100 g or more, for the purpose of improving the color difference between a pressed surface in an unused state and the surface after use.

### CITATION LIST

### Patent Literature

Patent Literature 1: Japanese Unexamined Patent Publication No. 2011-105626

### SUMMARY OF INVENTION

### Technical Problem

However, the technique described in the Patent Literature 1 improves the issue of a pressed surface shading into white; however, sufficient investigation was not conducted on the adherence of the cosmetic to the skin and color development upon application.

It is an object of the present invention to provide a solid powder cosmetic that exhibits excellent color development, has satisfactory feeling of use, and is capable of making the color difference between the surface color of an unused portion and the surface color of a used portion at a pressed surface sufficiently small, and to provide a method for producing the solid powder cosmetic.

### Solution to Problem

The inventors of the present invention conducted a thorough investigation in order to solve the problems described above, and as a result, the inventors found that with regard to a solid powder cosmetic containing a powder component and an oily component at particular proportions, when two particular oily components are incorporated as oily components, both the securement of color development and the feeling of use, and the improvement of hue at a pressed surface can be achieved in a well-balanced manner. Thus, the present invention was completed.

That is, the present invention provides a solid powder cosmetic containing a powder component and an oily component, in which the contents of the powder component and the oily component are 70% to 95% by mass and 5% to 30% by mass, respectively, based on the total amount of the solid powder cosmetic, and the oily component contains (A) a hardened castor oil fatty acid ester and (B) a heavy liquid isoparaffin.

According to the solid powder cosmetic according to the present invention, when the solid powder cosmetic has the above-described configuration, excellent color development is achieved, and satisfactory feeling of use is obtained. Also, even in a case in which the solid powder cosmetic is formed by a wet production method, the pressed surface shading into white, in other words, the pressed surface appearing whitish, can be sufficiently suppressed, and the color difference between the surface color of an unused portion and the surface color of a used portion can be made sufficiently small.

Furthermore, the solid powder cosmetic according to the present invention can be produced into a formed product that has excellent formability, does not easily cause powder flying and caking, and has sufficient impact resistance.

With regard to the solid powder cosmetic of the present invention, it is preferable that the content of the component (A) is 0.5% to 7.0% by mass based on the total amount of the solid powder cosmetic, and the content of the component (B) is 1.0% to 12.0% by mass based on the total amount of the solid powder cosmetic.

Furthermore, it is preferable that the solid powder cosmetic contains a plate-shaped powder as the powder component.

With regard to the solid powder cosmetic of the present invention, it is preferable that the mass ratio of the component (A) to the total amount of the oily component, [component (A)]/[oily component], is 1/10 to 3/5, and the mass ratio of the component (B) to the total amount of the oily component, [component (B)]/[oily component], is 1/10 to 3/5.

The present invention also provides a method for producing a solid powder cosmetic, the method including a step of mixing a cosmetic base material containing a powder component and an oily component with a dispersing medium containing a volatile solvent and preparing a slurry; and a step of compression-molding and drying the slurry charged into a vessel, in which the contents of the powder component and the oily component in the cosmetic base material are 70% to 95% by mass and 5% to 30% by mass, respectively, based on the total amount of the cosmetic base material, and the oily component contains (A) a hardened castor oil fatty acid ester and (B) a heavy liquid isoparaffin.

According to the method for producing a solid powder cosmetic according to the present invention, by forming a cosmetic base material having the above-described configuration by a wet production method, a solid powder cosmetic which can sufficiently suppress a pressed surface shading into white, exhibits excellent color development, and has satisfactory feeling of use, and in which the color difference between the surface color of an unused portion and the surface color of a used portion is sufficiently small, can be obtained. Furthermore, in the method for producing a solid powder cosmetic of the present invention, a formed product that does not easily cause powder flying and caking and has sufficient impact resistance, can be obtained.

With regard to the method for producing a solid powder cosmetic of the present invention, it is preferable that the content of the component (A) in the cosmetic base material is 0.5% to 7.0% by mass based on the total amount of the cosmetic base material, and the content of the component (B) in the cosmetic base material is 1.0% to 12.0% by mass based on the total amount of the cosmetic base material.

Furthermore, it is preferable that the cosmetic base material contains a plate-shaped powder as the powder component.

Furthermore, with regard to the cosmetic base material, it is preferable that the mass ratio of the component (A) to the total amount of the oily component, [component (A)]/[oily component], is 1/10 to 3/5, and the mass ratio of the component (B) to the total amount of the oily component, [component (B)]/[oily component], is 1/10 to 3/5.

### Advantageous Effects of Invention

According to the present invention, a solid powder cosmetic which exhibits excellent color development, and has satisfactory feeling of use, and in which the color difference between the surface color of an unused portion and the surface color of a used portion at a pressed surface can be made sufficiently small, and a method for producing the solid powder cosmetic can be provided.

### DESCRIPTION OF EMBODIMENTS

The solid powder cosmetic of the present embodiment contains a powder component and an oily component.

Regarding the powder component, any powder that is usually used in a cosmetic can be used without particular limitations, and for example, an extender powder, a white pigment, a colored pigment, and the like may be mentioned. The shape of the powder is also not particularly limited, and the powder may have a shape such as a spherical shape, a plate shape, or a needle shape; a particle size of a mist form, a microparticulate grade, or a pigment grade; and a particle structure such as a porous structure or a non-porous structure.

Specific examples of the powder component include extender pigments such as mica, synthetic mica, sericite, talc, kaolin, silicon carbide, barium sulfate, bentonite, smectite, aluminum oxide, silica, magnesium oxide, zirconium oxide, magnesium carbonate, calcium carbonate, chromium oxide, and aluminum magnesium hydroxide; white pigments such as titanium oxide and zinc oxide; organic powders such as a nylon powder, a polymethyl methacrylate powder, an acrylonitrile-methacrylic acid copolymer powder, a vinylidene chloride-methacrylic acid copolymer, a polyethylene powder, a polystyrene powder, an organopolysiloxane elastomer powder, a polymethylsilsesquioxane powder, a urethane powder, a wool powder, a silk powder, a cellulose powder, and an N-acyl lysine powder; composite powders such as a microparticulate titanium oxide-coated titanated mica, microparticulate titanium oxide-coated nylon, barium sulfate-coated titanated mica, titanium oxide-containing silica, and zinc oxide-containing silica; and metal soaps such as magnesium stearate, zinc myristate, aluminum stearate, and calcium stearate.

Examples of the colored pigment include inorganic colored pigments such as red iron oxide, yellow iron oxide, black iron oxide, cobalt oxide, chromium oxide, Ultramarine blue, Prussian blue, titanium oxide, and zinc oxide; organic colored pigments such as Red No. 228, Red No. 226, Blue No. 404, Red No. 202, and Yellow No. 4 aluminum lake; pearl pigments such as titanated mica, microparticulate titanium oxide-coated titanated mica, barium sulfate-coated titanated mica, fish scale guanine, bismuth oxychloride, and aluminum flakes; and natural colorants such as carmine and safflower; and the like.

These powder components are preferably hydrophobic-treated powders, from the viewpoints of color development and adherence. Regarding the hydrophobic treatment, higher fatty acids, metal soaps, fats and oils, waxes, silicone compounds, fluorine compounds, surfactants, and dextrin fatty acid esters may be mentioned. According to the present embodiment, from the viewpoints of the feeling of use and color development, a metal soap treatment is preferred, and a magnesium stearate treatment is more preferred.

Regarding the powder component, one kind thereof can be used alone, or two or more kinds thereof can be used in combination.

The average particle size of the powder component can be adjusted to 1 to 200 μm, and can be adjusted to 5 to 150 μm.

With regard to the solid powder cosmetic of the present embodiment, from the viewpoint of further enhancing color development and the feeling of use, it is preferable that the powder component includes a plate-shaped powder.

Regarding the plate-shaped powder, from the viewpoints of color development and the feeling of use, the average particle size is preferably 1.0 to 30.0 μm, and the aspect ratio is preferably 10 to 100, more preferably 15 to 90, and even more preferably 20 to 80. Meanwhile, the average particle size of the plate-shaped powder means the 50% median diameter according to a laser diffraction method. Furthermore, the aspect ratio means average particle size/average thickness.

Regarding the plate-shaped powder, for example, talc, mica, synthetic mica, sericite, silicic anhydride, kaolin, calcium carbonate, and a lustrous powder can be used. Incidentally, in regard to conventional solid powder cosmetics, when a lustrous powder is incorporated, powder flying is likely to occur, and the adherence tends to decrease. However, according to the solid powder cosmetic of the present embodiment, since the component (A) and component (B) that will be described below as oily components are incorporated, the occurrence of powder flying can be sufficiently suppressed, and also, adherence can be sufficiently secured.

The content of a plate-shaped powder in the solid powder cosmetic of the present embodiment is preferably 20% to 100% by mass, and more preferably 60% to 100% by mass, based on the total amount of the powder component. By adjusting the content of the plate-shaped powder to the above-described range, impact resistance can be further enhanced.

With regard to the solid powder cosmetic of the present embodiment, from the viewpoint that the occurrence of powder flying can be further suppressed, and impact resistance can be further enhanced, it is preferable that the powder component includes a metal soap.

Regarding the metal soap, those mentioned above can be used, and zinc myristate and aluminum stearate are preferred.

The content of the metal soap in the solid powder cosmetic of the present embodiment is preferably 0.5% to 10% by mass, and more preferably 1% to 5% by mass, based on the total amount of the powder component. By adjusting the content of the metal soap to the above-described range, the feeling of use is not impaired, the occurrence of powder flying can be further suppressed, and impact resistance can be further enhanced.

The content of the powder component in the solid powder cosmetic of the present embodiment can be adjusted to 70% to 95% by mass based on the total amount of the solid powder cosmetic, and from the viewpoints of formability and the feeling of use, it is preferable that the content is adjusted to 80% to 95% by mass.

According to the present embodiment, it is preferable that the oily component includes (A) a hardened castor oil fatty acid ester (may also be referred to as component (A)) and (B) a heavy liquid isoparaffin (may also be referred to as component (B)).

When the above-described component (A) and component (B) are incorporated in combination as oily components into a solid powder cosmetic including a powder component and an oily component, excellent color development and satisfactory feeling of use are obtained. Also, even in a case in which the solid powder cosmetic is formed by a wet production method, a pressed surface shading into white can be sufficiently suppressed, and the color difference between the surface color of an unused portion and the surface color of a used portion can be made sufficiently small. Furthermore, the solid powder cosmetic of the present embodiment can be produced into a formed product that has excellent formability, does not easily cause powder flying and caking, and has sufficient impact resistance.

Examples of the component (A) include isostearic acid hardened castor oil, hydroxystearic acid hardened castor oil, triisostearic acid hardened castor oil, stearic acid hardened castor oil, and lauric acid hardened castor oil. Regarding the component (A), one kind thereof can be used alone, or two or more kinds thereof can be used in combination.

From the viewpoint of making the feeling of use and the external appearance color of the pressed surface satisfactory, the content of the component (A) in the solid powder cosmetic of the present embodiment is preferably 0.5% to 7.0% by mass, more preferably 1.0% to 6.0% by mass, and even more preferably 1.5% to 5.0% by mass, based on the total amount of the solid powder cosmetic.

With regard to the solid powder cosmetic of the present embodiment, from the viewpoint of the feeling of use such as adherence, spreading, the difficulty in powder flying, and the difficulty in caking, the mass ratio of the component (A) to the total amount of the oily component, [component (A)]/[oily component], is preferably 1/10 to 3/5, and more preferably 1/8 to 1/3.

Regarding the component (B), any material that is used in cosmetics can be used without particular limitations. Regarding the heavy liquid isoparaffin, a mixture of a copolymer of isobutene and n-butene with a saturated type long-chained hydrocarbon having a hydrogenated side chain also containing a terminal double bond, can be used. Furthermore, regarding the heavy liquid isoparaffin, commercially available products such as PERLEAM 18, PERLEAM 24, and PERLEAM 46 (all manufactured by NOF CORPORATION, trade name) can be used.

According to the present embodiment, as the component (B), a heavy liquid isoparaffin having a dynamic viscosity at 98.9° C. of 300 to 4,700 mm²/s can be used.

Furthermore, regarding the component (B), from the viewpoints of the feeling of use, the difficulty in powder flying, impact resistance, and color development, a heavy liquid isoparaffn having a viscosity at 25° C. of 10,000 to 600,000 mPa-s is preferred, and a heavy liquid isoparaffin having a viscosity at 25° C. of 20,000 to 150,000 mPa-s is more preferred. Meanwhile, the viscosity is measured at 25° C. under the following conditions: in a case in which the viscosity of the heavy liquid isoparaffin is 20 to 100 mPa-s, using a BM type viscometer and rotor No. 2 at a speed of rotation of 60 rpm; in a case in which the viscosity is 10,000 to 100,000 mPa-s, using a BH type viscometer and rotor No. 6 at a speed of rotation of 10 rpm; and in a case in which the viscosity is 100,000 to 800,000 mPa-s, using a BH type viscometer and rotor No. 7 at a speed of rotation of 4 rpm. Meanwhile, for the viscometers and the rotors, products manufactured by Toki Sangyo Co., Ltd. can be used.

The content of the component (B) in the solid powder cosmetic of the present embodiment is preferably 1.0% to 12.0% by mass, more preferably 2.0% to 10.0% by mass, and even more preferably 3.0% to 8.0% by mass, based on the total amount of the solid powder cosmetic, from the viewpoints of color development and the feeling of use.

With regard to the solid powder cosmetic of the present embodiment, from the viewpoint of the feeling of use such as adherence, spreading, the difficulty in powder flying, and the difficulty in caking, the mass ratio of the component (B) to the total amount of the oily component, [component (B)]/[oily component], is preferably 1/10 to 3/5, and more preferably 1/4 to 1/2.

Regarding an oily component other than the component (A) and the component (B), for example, any oily component that is usually used in cosmetic products can be used without particular limitations, and irrespective of the origin such as an animal oil, a plant oil, or a synthetic oil, and of the nature such as a solid oil, a semi-solid oil, a liquid oil, or a volatile oil, oils and fats, hardened oils, fatty acids, higher alcohols, silicone oils, fluorine-based oils, lanolin derivatives, oily gelling agents, and the like can be used.

With regard to the solid powder cosmetic of the present embodiment, from the viewpoint of color development, it is preferable that the component (B) and the oily component other than the component (A) and the component (B) are oil agents having a refractive index of 1.47 to 1.60. Examples of an oil agent other than the component (A) and the component (B), which has such a refractive index, include silicone oils such as diphenylsiloxy phenyl trimethicone; cholesterol fatty acid esters such as cholesteryl hydroxystearate and cholesteryl oleate; and phytosteryl fatty acid esters such as phytosteryl oleate.

The solid powder cosmetic of the present embodiment can contain, in addition to the above-described components, components that are usually used in cosmetics, for example, an antiseptic agent, an oxidation inhibitor, a colorant, a thickening agent, a pH adjusting agent, a fragrance, an ultraviolet absorber, an ultraviolet scattering agent, a humectant, a chelating agent, an anti-inflammatory agent, a surfactant, and the like.

The solid powder cosmetic of the present embodiment is suitable as a makeup cosmetic for foundations, face colors, eye shadows, eyebrows, blushers, and the like.

Next, the method for producing a solid powder cosmetic of the present embodiment will be described.

The method for producing a solid powder cosmetic of the present embodiment includes a step of mixing a cosmetic base material containing a powder component and an oily component with a dispersing medium and preparing a slurry; and a step of compression-molding and drying the slurry charged into a vessel.

Regarding the powder component and the oily component, those mentioned above may be used, and the mixing amounts in the cosmetic base material can be adjusted to be similar to the preferred ranges in the solid powder cosmetic as described above. Furthermore, the composition other than the powder component and the oily component of the cosmetic base material can also be made similar to the preferred composition of the solid powder cosmetic as mentioned above.

The cosmetic base material according to the present embodiment can be prepared by, for example, a method including a step of mixing powder components and obtaining a first mixture; a step of mixing oily components and obtaining a second mixture; and a step of mixing the first mixture and the second mixture.

The step of obtaining a first mixture can be carried out using, for example, a Super Mixer, a Henschel Mixer, or the like, and if necessary, pulverization may be carried out using an atomizer or the like.

The step of obtaining a second mixture can be carried out using, for example, a Disper, a Homomixer, or the like, and the oily components can be mixed while heating the oily components at 60° C. to 80° C., and preferably 60° C. to 70° C.

The step of mixing the first mixture and the second mixture can be carried out using, for example, a Super Mixer, a Henschel Mixer, or the like, and if necessary, pulverization may be carried out using an atomizer or the like.

Regarding the preparation of a slurry, a method of adding a dispersing medium to the cosmetic base material obtainable as described above and mixing these, may be mentioned.

Regarding the dispersing medium, a volatile solvent can be used. Examples of the volatile solvent include light liquid isoparaffin, ethyl alcohol, acetone, isopropyl alcohol, and water.

The mixing proportions of the cosmetic base material and the dispersing medium can be adjusted to cosmetic base material dispersing medium=100:60 to 100:10 as a mass ratio, and from the viewpoint of formability, the mixing proportions are preferably 100:40 to 100:20.

Regarding the mixing of the cosmetic base material and the dispersing medium, for example, a method of kneading using a kneader, a universal stirrer, or the like may be mentioned. Furthermore, if necessary, the cosmetic base material and the dispersing medium can be mixed while heating.

In the step of compression-molding and drying the slurry charged into a vessel, the slurry obtained as described above is defoamed as necessary and is charged into a predetermined vessel, and then this slurry is compression-molded by suction compression-molding or the like. Subsequently, the resultant can be dried appropriately in a dryer.

Regarding the predetermined vessel, an inner tray such as a metal tray or a resin tray, or the like may be mentioned.

The solid powder cosmetic according to the present embodiment is obtained through the above-described steps.

### EXAMPLES

Hereinafter, the present invention will be described in more detail by way of Examples; however, the technical scope of the present invention is not intended to be limited by these Examples. Meanwhile, the numerical values in the tables represent the contents (% by mass) based on the total amount of the cosmetic base material (sum of components other than a volatile solvent). With regard to a volatile solvent, the numerical value represents the proportion (parts by mass) with respect to 100 parts by mass of the total amount of the cosmetic base material (sum of components other than the volatile solvent).

Prior to Examples, the evaluation methods employed in the various Examples will be described.

(1) Color Difference

The surface of a cosmetic was repeatedly scraped for 30 times with a cosmetic tip, and the color difference ΔE′ between the surface color of a used portion and the surface color of an unused portion was measured using a color difference meter, CR-400 (manufactured by KONICA MINOLTA, INC.). The color difference was determined according to the following evaluation criteria.

[Evaluation Criteria]

A: ΔE′<1.5

B: 1.5≤ΔE′<2

C: 2≤ΔE*<2.5

D: 2.5≤ΔE*

(2) Excellence of Feeling of Use and Color Development

Twenty expert panels for cosmetic product evaluation were asked to use solid powder cosmetics of Examples and Comparative Examples, and each of the expert panels conducted a 5-grade evaluation for the “spreading” and “adherence” as the feeling of use, and the “excellence of color development” according to the following evaluation criteria and assigned a score to each sample. Furthermore, the average points of the scores of all the panels were determined according to the following criteria.

[Score: Evaluation Criteria]

5 points: Very satisfactory

4 points: Satisfactory

3 points: Ordinary

2 points: Slightly poor

1 point: Poor

[Determination Criteria (Average Point of Scores)]

A: More than or equal to 4

B: More than or equal to 3 and less than 4

C: More than or equal to 2 and less than 3

D: Less than 2

(3) Powder Flying

A solid powder cosmetic thus obtained was swept for 30 times with a cosmetic tip, and the amount of powder that had fallen without adhering was weighed and determined according to the following determination criteria.

[Determination Criteria]

A: ¼ or less of the amount of adherence

B: More than ¼ and less than or equal to ½ of the amount of adherence

\vec{C}: More than 1/2 and less than or equal to equivalence to the amount of adherence

D: More than equivalence to the amount of adherence

(4) Formability For a formed article obtained by filling an inner tray with a slurry, subsequently compression-molding the slurry, and drying the slurry, the presence or absence of cracks, peeling, fissures, and the like generated on the surface of the formed article was observed by visual inspection, and the level of generation thereof was determined according to the following 4-grade evaluation criteria.

[Evaluation Criteria]

A: No change.

B: Some cracks, peeling, or fissures are recognized.

C: Cracks, peeling, or fissures are recognized.

D: Severe cracks, peeling, or fissures are recognized.

(5) Impact Resistance

Each solid powder cosmetic was incorporated into a cosmetic compact, the cosmetic compact was dropped five times from a height of 50 cm in the horizontal direction onto a tile, and the surface state was observed. The surface state was determined according to the following evaluation criteria.

[Evaluation Criteria]

A: No change.

B: Some cracks and peeling are recognized.

C: Cracks and peeling are recognized.

D: Severe cracks and peeling are recognized.

(6) Caking

The solid powder cosmetic was repeatedly scraped for 30 times in the same direction with a cosmetic tip, the surface of a used portion was observed by visual inspection, and the surface was determined according to the following evaluation criteria.

[Evaluation Criteria]

A: Caking is not observed.

B: Caking is observed; however, caking is resolved in the middle of scraping for 30 times.

C: Caking is observed; however, there is no problem in usability.

D: Caking is observed, and there is a problem in usability.

### Examples 1 to 13 and Comparative Examples 1 to 6

Eye shadows having the compositions shown in Tables 1 to 3 were prepared by the following production method, and the above-described evaluations were carried out. The results are presented together in Tables 1 to 3.

<Production Method>

Oily components were heated to 60° C. to 80° C. and mixed, and mixture I was obtained. Powder components were uniformly dispersed with a Henschel Mixer, and mixture II was obtained. I was added to II, the mixture was uniformly dispersed with a Henschel Mixer, and a cosmetic base material was obtained. To 100 parts by mass of this cosmetic base material, 30 parts by mass of light liquid isoparaffin as a volatile solvent was added, and the mixture was slurrified by kneading. Subsequently, an inner tray (metal tray) was filled with the slurry, the slurry was compression-molded, and then the solvent was removed by drying the slurry. Thereby, an eye shadow was produced.

TABLE 1

| | | | Example | | | | | | |
|---|---|---|---|---|---|---|---|---|---|
| | | | 1 | 2 | 3 | 4 | 5 | 6 | 7 |
| Oily | Component (A) | Isostearic acid | 2.0 | — | — | — | 2.0 | 2.0 | — |
| component | | hardened castor oil | | | | | | | |
| | | Hydroxystearic acid | — | 2.0 | — | — | — | — | 2.0 |
| | | hardened castor oil | | | | | | | |
| | | Triisostearic acid | — | — | 2.0 | — | — | — | — |
| | | hardened castor oil | | | | | | | |
| | | Stearic acid | — | — | — | 2.0 | — | — | — |
| | | hardened castor oil | | | | | | | |
| | Component (B) | Heavy liquid isoparaffin A | — | — | — | — | 6.0 | — | — |
| | | Heavy liquid isoparaffin B | 6.0 | 6.0 | 6.0 | 6.0 | — | — | 6.0 |
| | | Heavy liquid isoparaffin C | — | — | — | — | — | 6.0 | — |
| | Others | Diphenylsiloxy phenyl | 4.0 | 4.0 | 4.0 | 4.0 | 4.0 | 4.0 | — |
| | | trimethicone | | | | | | | |
| | | Dimethylpolysiloxane | — | — | — | — | — | — | 4.0 |
| | | Diisostearyl malate | 3.0 | 3.0 | 3.0 | 3.0 | 3.0 | 3.0 | 3.0 |

| | | | | | | | | |
|---|---|---|---|---|---|---|---|---|
| Powder | Talc A | 20.0 | 20.0 | 20.0 | 20.0 | 20.0 | 20.0 | 20.0 |
| component | Synthetic mica A | 30.0 | 30.0 | 30.0 | 30.0 | 30.0 | 30.0 | 30.0 |
| | Boron nitride | 5.0 | 5.0 | 5.0 | 5.0 | 5.0 | 5.0 | 5.0 |
| | Zinc myristate | 2.0 | 2.0 | 2.0 | 2.0 | 2.0 | 2.0 | 2.0 |
| | Mg stearate-treated red iron oxide | 4.0 | 4.0 | 4.0 | 4.0 | 4.0 | 4.0 | 4.0 |
| | Mg stearate-treated black iron oxide | 2.0 | 2.0 | 2.0 | 2.0 | 2.0 | 2.0 | 2.0 |
| | Mg stearate-treated yellow iron oxide | 4.0 | 4.0 | 4.0 | 4.0 | 4.0 | 4.0 | 4.0 |
| | Iron oxide-coated titanated mica | 10.0 | 10.0 | 10.0 | 10.0 | 10.0 | 10.0 | 10.0 |
| | Mica A | Balance | Balance | Balance | Balance | Balance | Balance | Balance |
| Volatile | Light liquid isoparaffin | 30 | 30 | 30 | 30 | 30 | 30 | 30 |
| solvent | | | | | | | | |
| Evaluation | Color difference between | A | A | A | A | A | A | B |
| | appearance color and inner color | | | | | | | |
| | Feeling of use (spreading, adherence) | A | A | A | A | B | B | B |
| | Excellence of color development | A | A | B | A | A | A | A |
| | Absence of powder flying | A | A | A | A | B | A | A |
| | Formability | A | A | A | A | A | A | A |
| | Impact resistance | A | A | A | A | A | A | A |
| | Caking | A | A | A | A | A | B | B |

TABLE 2

| | | | Example | | | | | |
|---|---|---|---|---|---|---|---|---|
| | | | 8 | 9 | 10 | 11 | 12 | 13 |
| Oily | Component (A) | Isostearic acid | 1.0 | 3.0 | — | — | 2.0 | 2.0 |
| component | | hardened castor oil | | | | | | |
| | | Hydroxystearic acid | — | — | 2.0 | 2.0 | — | — |
| | | hardened castor oil | | | | | | |
| | Component (B) | Heavy liquid isoparaffin B | 2.0 | 6.0 | 6.0 | 6.0 | 6.0 | 6.0 |
| | Olliers | Diphenyisiloxy phenyl | 4.0 | 4.0 | 4.0 | 4.0 | 4.0 | 4.0 |
| | | trimethicone | | | | | | |
| | | Diisostearyl malate | 2.0 | 4.0 | 3.0 | 3.0 | 3.0 | 3.0 |

| | | | | | | | |
|---|---|---|---|---|---|---|---|
| Powder | Talc A | 20.0 | 10.0 | 20.0 | 20.0 | — | — |
| component | Talc B | — | — | — | — | 20.0 | — |
| | Talc C | — | — | — | — | — | 20.0 |
| | Synthetic mica A | 30.0 | 30.0 | 30.0 | 30.0 | — | — |
| | Synthetic mica B | — | — | — | — | 30.0 | — |
| | Mica B | — | — | — | — | — | 30.0 |
| | Boron nitride | 5.0 | 5.0 | 5.0 | 5.0 | 5.0 | 5.0 |
| | Zinc myristate | 2.0 | 2.0 | 2.0 | 2.0 | 2.0 | 2.0 |
| | Mg stearate-treated red iron oxide | 4.0 | 4.0 | — | — | 4.0 | 4.0 |
| | Mg stearate-treated black | 2.0 | 2.0 | — | — | 2.0 | 2.0 |
| | iron oxide | | | | | | |
| | Mg stearate-treated yellow | 4.0 | 4.0 | — | — | 4.0 | 4.0 |
| | iron oxide | | | | | | |
| | Dimethicone-treated red iron oxide | — | — | 4.0 | — | — | — |
| | Dimethicone-treated black iron oxide | — | — | 2.0 | — | — | — |
| | Dimethicone-treated yellow iron oxide | — | — | 4.0 | — | — | — |
| | Red iron oxide | — | — | — | 4.0 | — | — |
| | Black iron oxide | — | — | — | 2.0 | — | — |
| | Yellow iron oxide | — | — | — | 4.0 | — | — |
| | Iron oxide-coated titanated mica | 20.0 | 10.0 | 20.0 | 20.0 | — | — |
| | Mica A | Balance | Balance | Balance | Balance | Balance | Balance |
| Volatile | Light liquid isoparaffin | 30 | 30 | 30 | 30 | 30 | 30 |
| solvent | | | | | | | |
| Evaluation | Color difference between appearance | B | A | A | A | A | A |
| | color and inner color | | | | | | |
| | Feeling of use (spreading, adherence) | B | B | B | B | B | B |
| | Excellence of color development | B | A | A | B | B | B |
| | Absence of powder flying | A | A | B | B | A | C |
| | Formability | A | B | A | A | B | B |
| | Impact resistance | A | A | A | A | A | B |
| | Caking | A | B | A | A | B | A |

TABLE 3

| | | | Comparative Example | | | | | |
|---|---|---|---|---|---|---|---|---|
| | | | 1 | 2 | 3 | 4 | 5 | 6 |
| Oily | Component (A) | Isostearic acid | — | — | 2.0 | 2.0 | — | — |
| component | | hardened castor oil | | | | | | |
| | | Hydroxystearic acid | — | — | — | — | 0.5 | 4.0 |
| | | hardened castor oil | | | | | | |
| | Component (B) | Heavy liquid isoparaffin B | 6.0 | 6.0 | — | — | 1.0 | 12.0 |
| | Others | Liquid isoparaffin | — | — | — | 6.0 | — | — |
| | | Petrolatum | — | 2.0 | — | — | — | — |
| | | Diphenylsiloxy phenyl | 4.0 | 4.0 | 4.0 | 4.0 | 1.0 | 10.0 |
| | | trimethicone | | | | | | |
| | | Diisostearyl malate | 3.0 | 3.0 | 7.0 | 3.0 | 0.5 | 9.0 |

| | | | | | | | |
|---|---|---|---|---|---|---|---|
| Powder | Talc A | 20.0 | 20.0 | 20.0 | 20.0 | 20.0 | 10.0 |
| component | Synthetic mica A | 30.0 | 30.0 | 30.0 | 30.0 | 30.0 | 15.0 |
| | Boron nitride | 5.0 | 5.0 | 5.0 | 5.0 | 5.0 | 5.0 |
| | Zinc myristate | 2.0 | 2.0 | 2.0 | 2.0 | 2.0 | 2.0 |
| | Mg stearate-treated red iron oxide | 4.0 | 4.0 | 4.0 | 4.0 | 4.0 | 4.0 |
| | Mg stearate-treated black iron oxide | 2.0 | 2.0 | 2.0 | 2.0 | 2.0 | 2.0 |
| | Mg stearate-treated yellow iron oxide | 4.0 | 4.0 | 4.0 | 4.0 | 4.0 | 4.0 |
| | Iron oxide-coated titanated mica | 10.0 | 10.0 | 10.0 | 10.0 | 10.0 | 10.0 |
| | Mica A | Balance | Balance | Balance | Balance | Balance | Balance |
| Volatile | Light liquid isoparaffin | 30 | 30 | 30 | 30 | 30 | 30 |
| solvent | | | | | | | |
| Evaluation | Color difference between appearance | D | D | C | B | B | C |
| | color and inner color | | | | | | |
| | Feeling of use (spreading, adherence) | C | B | C | C | C | C |
| | Excellence of color development | C | C | D | D | D | C |
| | Absence of powder flying | C | C | D | D | D | B |
| | Formability | A | A | A | A | A | D |
| | Impact resistance | B | A | C | C | D | A |
| | Caking | A | A | B | A | A | D |

In Tables 1 to 3, the details of the various components are as follows.

[Oily Component]

Component (A)

Isostearic acid hardened castor oil: Castolide MIS-P (manufactured by National Mimatsu Co., Ltd.)

Hydroxystearic acid hardened castor oil: Technol MH (manufactured by Yokozeki Oil & Fat Industries Co., Ltd.)

Triisostearic acid hardened castor oil: Castolide TIS-P (manufactured by National Mimatsu Co., Ltd.)

Stearic acid hardened castor oil: Castolide MS (manufactured by National Mimatsu Co., Ltd.)

Component (B)

Heavy liquid isoparaffin A: PERLEAM 18 (manufactured by NOF CORPORATION) (28,000 mPa·s@25° C.)

Heavy liquid isoparaffin B: PERLEAM 24 (manufactured by NOF CORPORATION) (86,500 mPa·s@25° C.)

Heavy liquid isoparaffin C: PERLEAM 46 (manufactured by NOF CORPORATION) (580,000 mPa·s@25° C.)

(Others)

Liquid isoparaffin: PERLEAM 6 (manufactured by NOF CORPORATION) (37.5 mPa·s@25° C.)

Diphenylsiloxy phenyl trimethicone: KF-56A (manufactured by Shin-Etsu Chemical Co., Ltd.)

Dimethylpolysiloxane: KF-96-100CS (manufactured by Shin-Etsu Chemical Co., Ltd.)

Meanwhile, the viscosity was measured at 25° C., under the following conditions: in a case in which the viscosity was 20 to 100 mPa-s, using a BM type viscometer and a rotor No. 2 at a speed of rotation of 60 rpm; in a case in which the viscosity was 10,000 to 100,000 mPa-s, using a BH type viscometer and a rotor No. 6 at a speed of rotation of 10 rpm; and in a case in which the viscosity was 100,000 to 800,000 mPa-s, using a BH type viscometer and a rotor No. 7 at a speed of rotation of 4 rpm. The above-described viscometers and rotors were products manufactured by Toki Sangyo Co., Ltd.

[Powder Component]

Talc A: Talc (average particle size 25 μm, aspect ratio 30)

Talc B: Talc (average particle size 9.5 μm, aspect ratio 50)

Talc C: Talc (average particle size 30 μm, aspect ratio 15)

Synthetic mica A: Synthetic mica (average particle size 20 μm, aspect ratio 60)

Synthetic mica B: Synthetic mica (average particle size 12 μm, aspect ratio 25)

Mica A: Mica (average particle size 2 μm)

Mica B: Mica (average particle size 26 μm, aspect ratio 65)

Boron nitride: Average particle size 7 μm

Iron oxide-coated titanated mica: Average particle size 21 μm

As shown in Tables 1 and 2, the solid powder cosmetics obtainable in Examples to 13 were rated as “B” or “A” with regard to the “Color difference between appearance color and inner color”, “Feeling of use (spreading, adherence)”, and “Excellence of color development”. Furthermore, the solid powder cosmetics obtainable in Examples 1 to 13 were rated as “B” or “A” with respect to “Absence of powder flying”, “Formability”, “Impact resistance”, and “Caking”, except that the “Absence of powder flying” of the solid powder cosmetic of Example 13 was rated as “C”.

### Example 14: Eye Shadow

| | |
|---|---|
| | (Mixing proportion |
| (Component) | (% by mass)) |

| | | |
|---|---|---|
| 1. | Isostearic acid hardened castor oil | 2.00 |
| 2. | Heavy liquid isoparaffin A | 4.00 |
| 3. | Diphenylsiloxy phenyl trimethicone | 8.00 |
| 4. | Diisostearyl malate | 3.00 |
| 5. | Talc A | 20.00 |
| 6. | Synthetic mica A | 25.00 |
| 7. | Boron nitride | 5.00 |
| 8. | Zinc myristate | 2.00 |
| 9. | Red iron oxide | 0.10 |
| 10. | Yellow iron oxide | 0.30 |
| 11. | Black iron oxide | 0.25 |
| 12. | Titanated mica A | 15.00 |
| 13. | Titanium oxide-coated glass | 15.00 |
| 14. | Mica A | Balance |

Volatile solvent: 20 parts by mass of light liquid isoparaffin with respect to 100 parts by mass of the sum of the above-described components 1 to 14

The details of the components described above are similar to those previously mentioned above, except for the components shown below.

Titanated mica A: Titanated mica (average particle size 53 μm) Titanium oxide-coated glass: Average particle size 80 μm, aspect ratio 80

<Production Method>

Components 1 to 4 were heated to 60° C. to 80° C. and mixed, and mixture I was obtained. Components 5 to 14 were uniformly dispersed with a Henschel Mixer, and mixture II was obtained. I was added to 11, the mixture was uniformly dispersed with a Henschel Mixer, and a cosmetic base material was obtained. To this cosmetic base material, the above-described volatile solvent was added, and the mixture was slurrified by kneading. Subsequently, an inner tray (metal tray) was filled with the slurry, the slurry was compression-molded, and then the solvent was removed by drying the slurry. Thereby, an eye shadow was produced.

<Evaluation>

The sample of eye shadow thus obtained was subjected to evaluations similar to those described above, and it was verified that the sample was rated as “B” or “A” for all the items.

### Example 15: Eyebrow

| | |
|---|---|
| | (Mixing proportion |
| (Component) | (% by mass)) |

| | | |
|---|---|---|
| 1. | Hydroxystearic acid hardened castor oil | 1.50 |
| 2. | Heavy liquid isoparaffin A | 4.00 |
| 3. | Diphenylsiloxy phenyl trimethicone | 3.50 |
| 4. | Diisostearyl malate | 3.00 |
| 5. | Talc A | 20.00 |
| 6. | Synthetic mica A | 30.00 |
| 7. | Boron nitride | 5.00 |
| 8. | Zinc myristate | 2.00 |
| 9. | Mg stearate-treated red iron oxide | 8.00 |
| 10. | Mg stearate-treated black iron oxide | 10.00 |
| 11. | Mg stearate-treated yellow iron oxide | 4.00 |
| 12. | Iron oxide-coated titanated mica | 10.00 |
| 13. | Mica A | Balance |

Volatile solvent: 40 parts by mass of light liquid isoparaffin with respect to 100 parts by mass of the sum of the above-described components 1 to 14

The details of the above-described components are similar to those previously mentioned above.

<Production Method>

Components 1 to 4 were heated to 60° C. to 80° C. and mixed, and mixture I was obtained. Components 5 to 13 were uniformly dispersed with a Henschel Mixer, and mixture II was obtained. I was added to II, the mixture was uniformly dispersed with a Henschel Mixer, and a cosmetic base material was obtained. To this cosmetic base material, the above-described volatile solvent was added, and the mixture was slurrified by kneading. Subsequently, an inner tray (metal tray) was filled with the slurry, the slurry was compression-molded, and then the solvent was removed by drying the slurry. Thereby, an eyebrow was produced.

<Evaluation>

The sample of eyebrow thus obtained was subjected to evaluations similar to those described above, and it was verified that the sample was rated as “B” or “A” for all the items.

### Example 16: Blusher

| | |
|---|---|
| | (Mixing proportion |
| (Component) | (% by mass)) |

| | | |
|---|---|---|
| 1. | Isostearic acid hardened castor oil | 1.00 |
| 2. | Heavy liquid isoparaffin A | 2.00 |
| 3. | Diphenylsiloxy phenyl trimethicone | 4.00 |
| 4. | Diisostearyl malate | 3.00 |
| 5. | Talc A | 30.00 |
| 6. | Synthetic mica A | 35.00 |
| 7. | Boron nitride | 5.00 |
| 8. | Zinc myristate | 2.00 |
| 9. | Red iron oxide | 0.25 |
| 10. | Yellow iron oxide | 0.25 |
| 11. | Ultramarine blue | 0.75 |
| 12. | Red 226 | 0.75 |
| 13. | Titanated mica B | 15.00 |
| 14. | Mica A | Balance |

As a volatile solvent, 30 parts by mass of light liquid isoparaffin was mixed in with respect to 100 parts by mass of the sum of the above-described components 1 to 14.

The details of the above-described components are similar to those previously mentioned above, except for the component shown below.

Titanated mica B: Titanated mica (average particle size 21 μm)

<Production Method>

Components 1 to 4 were heated to 60° C. to 80° C. and mixed, and mixture I was obtained. Components 5 to 14 were uniformly dispersed with a Henschel Mixer, and mixture II was obtained. I was added to II, the mixture was uniformly dispersed with a Henschel Mixer, and a cosmetic base material was obtained. To this cosmetic base material, the above-described volatile solvent was added, and the mixture was slurrified by kneading. Subsequently, an inner tray (metal tray) was filled with the slurry, the slurry was compression-molded, and then the solvent was removed by drying the slurry. Thereby, a blusher was produced.

<Evaluation>

The sample of blusher thus obtained was subjected to evaluations similar to those described above, and it was verified that the sample was rated as “B” or “A” for all the items.

### Example 17: Foundation

| | |
|---|---|
| | (Mixing proportion |
| (Component) | (% by mass)) |

| | | |
|---|---|---|
| 1. | Isostearic acid hardened castor oil | 2.00 |
| 2. | Heavy liquid isoparaffin A | 2.00 |
| 3. | Diphenylsiloxy phenyl trimethicone | 4.00 |
| 4. | Diisostearyl malate | 2.00 |
| 5. | Talc A | 30.00 |
| 6. | Synthetic mica A | 30.00 |
| 7. | Boron nitride | 5.00 |
| 8. | Zinc myristate | 2.00 |
| 9. | Red iron oxide | 1.50 |
| 10. | Yellow iron oxide | 1.50 |
| 11. | Black iron oxide | 0.20 |
| 12. | Titanium oxide | 2.50 |
| 13. | Microparticulate titanium oxide | 10.00 |
| 14. | Mica A | Balance |

As a volatile solvent, 40 parts by mass of light liquid isoparaffin was mixed in with respect to 100 parts by mass of the sum of the above-described components 1 to 14.

The details of the above-described components are similar to those previously mentioned above, except for the component shown below.

Microparticulate titanium oxide: Average particle size 7 μm, aspect ratio 4

<Production Method>

Components 1 to 4 were heated to 60° C. to 80° C. and mixed, and mixture I was obtained. Components 5 to 14 were uniformly dispersed with a Henschel Mixer, and mixture II was obtained. I was added to II, the mixture was uniformly dispersed with a Henschel Mixer, and a cosmetic base material was obtained. To this cosmetic base material, the above-described volatile solvent was added, and the mixture was slurrified by kneading. Subsequently, an inner tray (metal tray) was filled with the slurry, the slurry was compression-molded, and then the solvent was removed by drying the slurry. Thereby, a blusher was produced.

<Evaluation>

The sample of foundation thus obtained was subjected to evaluations similar to those described above, and it was verified that the sample was rated as “B” or “A” for all the items.

1. A solid powder cosmetic containing a powder component and an oily component,
wherein the contents of the powder component and the oily component are 70% to 95% by mass and 5% to 30% by mass, respectively, based on the total amount of the solid powder cosmetic,
the oily component contains (A) a hardened castor oil fatty acid ester and (B) a heavy liquid isoparaffin,
the content of the component (A) is 0.5% to 7.0% by mass based on the total amount of the solid powder cosmetic, and
the content of the component (B) is 1.0% to 12.0% by mass based on the total amount of the solid powder cosmetic.

2. (canceled)

3. The solid powder cosmetic according to claim 1, containing a plate-shaped powder having an aspect ratio of 10 to 100, as the powder component.

4. The solid powder cosmetic according to claim 1, wherein the mass ratio of the component (A) to the total amount of the oily component, [component (A)]/[oily component], is 1/10 to 3/5, and
the mass ratio of the component (B) to the total amount of the oily component, [component (B)]/[oily component], is 1/10 to 3/5.

5. A method for producing a solid powder cosmetic, the method comprising:
mixing a cosmetic base material containing a powder component and an oily component with a dispersing medium containing a volatile solvent and preparing a slurry; and
compression-molding and drying the slurry charged into a vessel,
wherein the contents of the powder component and the oily component in the cosmetic base material are 70% to 95% by mass and 5% to 30% by mass, respectively, based on the total amount of the cosmetic base material,
the oily component contains (A) a hardened castor oil fatty acid ester and (B) a heavy liquid isoparaffin,
the content of the component (A) in the cosmetic base material is 0.5% to 7.0% by mass based on the total amount of the cosmetic base material, and
the content of the component (B) in the cosmetic base material is 1.0% to 12.0% by mass based on the total amount of the cosmetic base material.

6. (canceled)

7. The method for producing a solid powder cosmetic according to claim 5, wherein the cosmetic base material contains a plate-shaped powder having an aspect ratio of 10 to 100, as the powder component.

8. The method for producing a solid powder cosmetic according to claim 5, wherein in the cosmetic base material, the mass ratio of the component (A) to the total amount of the oily component, [component (A)]/[oily component], is 1/10 to 3/5, and the mass ratio of the component (B) to the total amount of the oily component, [component (B)]/[oily component], is 1/10 to 3/5.

9. The solid powder cosmetic according to claim 1, wherein the content of the component (A) is 1.5% to 5.0% by mass based on the total amount of the solid powder cosmetic,
the content of the component (B) is 3.0% to 8.0% by mass based on the total amount of the solid powder cosmetic,
the mass ratio of the component (A) to the total amount of the oily component, [component (A)]/[oily component], is 1/8 to 1/3, and
the mass ratio of the component (B) to the total amount of the oily component, [component (B)]/[oily component], is 1/4 to 1/2.

10. The solid powder cosmetic according to claim 1, wherein the oily component contains oil agents having a refractive index of 1.47 to 1.60, as an oily component other than the component (A) and the component (B).

11. The solid powder cosmetic according to claim 10, wherein the oil agents is diphenylsiloxy phenyl trimethicone.

12. The solid powder cosmetic according to claim 10, wherein the content of the oil agents is 3.5% by mass or more, based on the total amount of the solid powder cosmetic.

13. The solid powder cosmetic according to claim 10, wherein the content of the oil agents is 3.5% to 8% by mass based on the total amount of the solid powder cosmetic.

14. The solid powder cosmetic according to claim 10, wherein the oil agents is diphenylsiloxy phenyl trimethicone, and the content of the oil agents is 3.5% to 8% by mass or more, based on the total amount of the solid powder cosmetic.

15. The method for producing a solid powder cosmetic according to claim 5, wherein the content of the component (A) is 1.5% to 5.0% by mass based on the total amount of the cosmetic base material,
the content of the component (B) is 3.0% to 8.0% by mass based on the total amount of the cosmetic base material,
the mass ratio of the component (A) to the total amount of the oily component, [component (A)]/[oily component], is 1/8 to 1/3, and
the mass ratio of the component (B) to the total amount of the oily component, [component (B)]/[oily component], is 1/4 to 1/2.

16. The method for producing a solid powder cosmetic according to claim 5, wherein the cosmetic base material contains oil agents having a refractive index of 1.47 to 1.60, as an oily component other than the component (A) and the component (B).

17. The method for producing a solid powder cosmetic according to claim 16, wherein the oil agents is diphenylsiloxy phenyl trimethicone.

18. The method for producing a solid powder cosmetic according to claim 16, wherein the content of the oil agents is 3.5% by mass or more, based on the total amount of the cosmetic base material.

19. The method for producing a solid powder cosmetic according to claim 16, wherein the content of the oil agents is 3.5% to 8% by mass based on the total amount of the cosmetic base material.

20. The method for producing a solid powder cosmetic according to claim 16, wherein the oil agents is diphenylsiloxy phenyl trimethicone, and the content of the oil agents is 3.5% to 8% by mass based on the total amount of the cosmetic base material.
